(19) United States
(12) Patent Application Publication
(10) Pub. No.: US 2007/0020249 A1
(43) Pub. Date: Jan. 25, 2007

(54) COMPOSITIONS FOR PREVENTION AND TREATEMENT OF SYMPTOMS OF GASTROINTESTINAL DISTRESS

(76) Inventor: Bernard William Downs, Lederach, PA (US)

Correspondence Address: PAULEY PETERSEN & ERICKSON, 2800 WEST HIGGINS ROAD, SUITE 365, HOFFMAN ESTATES, IL, 60195, US

(21) Appl. No.: 11/380,894

(22) Filed: Apr. 28, 2006

Related U.S. Application Data
(60) Provisional application No. 60/675,708, filed on Apr. 29, 2005.

(51) Int. Cl. A61K 35/74; A61K 31/695; A61K 38/47; A61K 31/555

(52) U.S. Cl. 424/93.45; 424/94.61; 514/63; 514/184

(57) ABSTRACT

A composition for the treatment or prevention of gastro-intestinal distress associates with the consumption of food products containing sugar alcohols includes at least one enzyme, at least one probiotic organism and simethicone. The composition may be consumed immediately prior to but not more than 20 minutes prior to consuming a sugar alcohol-containing food product in order to reduce the severity of at least one gastro-intestinal stress condition associated with sugar alcohol consumption by at least about 50%.

This application claims the benefit of U.S. Provisional Application 60/678,705 filed on 28 April 2005.

### FIELD OF THE INVENTION

The invention relates to compositions and methods for prevention or treatment of symptoms of gastrointestinal distress, including, but not limited to, diarrhea, loose or watery stools, inflammation, stomach and abdominal gas and/or pain, stomach rumblings, excessive belching, colic, flatulence, bloating, nausea and motion sickness. More particularly, the invention relates to compositions for prevention or treatment of symptoms of gastrointestinal distress and other symptoms caused by sugar alcohols, including, but not limited to, diarrhea, loose or watery stools, inflammation, stomach and abdominal gas and/or pain, stomach rumblings, excessive belching, colic, flatulence, bloating, nausea, motion sickness, heart burn and headaches.

### BACKGROUND OF THE INVENTION

The escalating health crisis in modernized industrial societies has been attributed to poor lifestyle choices hallmarked by the consumption of refined processed foods. In recent years, the consumption of sugar-free and no- and low-carbohydrate products has experienced a dramatic increase, largely driven by the dietary demands of a community of people comprised mostly of diabetics, overweight/obese individuals, and people trying to reduce refined carbohydrate consumption to lower and/or manage the incidence and severity of various carbohydrate allergies and sensitivities, insulin resistant disorders such as Syndrome X, and related disorders such as obesity, diabetes, hypertension and other cardiovascular diseases.

The most popular sweetening ingredients used to lower the “net” carbohydrate impact in sweetened foodstuffs are sugar alcohols. “Net” carbohydrates is a term used in recent years to identify the impact of certain carbohydrates on the body and is generally defined as the total amount of carbohydrates in a product with the fiber and sugar alcohols subtracted from this total amount. Fiber and sugar alcohols do not affect the body in the same way as other carbohydrates. Sugar alcohols are used in no- and low-carbohydrate candies, snacks, treats, beverages and food products to reduce the glycemic impact on insulin and diminish catalysis of insulin resistant conditions such as Syndrome X. The glycemic index is a ranking of carbohydrates based on their immediate effect on blood glucose levels. Carbohydrates that break down quickly have the highest glycemic indexes.

In addition, sugar alcohols, also known as polyols, are used as bulking agents. Sugar alcohols occur naturally in foods and come from plant products such as fruits and berries. As a sugar substitute, they provide fewer calories than regular sugar, having approximately 1.5 to 3 calories per gram compared to 4 calories per gram, and do not cause tooth decay associated with regular sugar. They can also be used to improve the glycemic load index of carbohydrates since they are converted to glucose more slowly, require little or no insulin to be metabolized, and don't cause any sudden increases in blood sugar. Thus, they offer a significant potential both as a weight control aid and to improve glycemic control, both of which are benefits that could be particularly useful for diabetics.

Other sugar substitutes exist such as artificial sweeteners like saccharin (marketed under the brand name Sweet & Low®). Several differences exist between the two types of sugar substitutes, i.e. sugar alcohols and artificial sweeteners. For one, artificial sweeteners have no calories whereas sugar alcohols contain about 2.6 calories per gram. Secondly, artificial sweeteners do not contain carbohydrates so they do not cause blood sugar levels to elevate whereas sugar alcohols have some effect on blood sugar. Third, complaints about flavor have been noted for artificial sweeteners; sugar alcohols are consistently more palatable. Overall, both types of sweeteners can be useful in diabetes management when used properly. However, concerns about deleterious effects on health have been reported for artificial sweeteners, making them less attractive to consumers.

The most common sugar alcohols are mannitol, sorbitol, xylitol, lactitol, isomalt, maltitol and hydrogenated starch hydrolysates (HSH). Although they are rarely used in foods prepared at home, they are often used in processed foods. Food products labeled “sugar-free”, including hard candies, cookies, chewing gums, soft drinks, throat lozenges, toothpaste and mouthwash, use these sugar alcohols. The chemical structure of these sugar alcohols delays breakdown in the stomach, allowing the molecules to pass intact into the intestines where they promote hygroscopicity, significantly increasing fluid volume and pressure in the gastrointestinal (GI) tract.

A number of adverse gastrointestinal effects have been reported with the use of sugar alcohols including diarrhea, loose or watery stools, inflammation, stomach and abdominal gas and/or pain, stomach rumblings, excessive belching, colic, flatulence, bloating, nausea and motion sickness in a dose-related manner. In fact, food products containing sugar alcohols often carry a label warning, indicating that consumption of the product can produce laxative effects. Some Type I diabetics have also found that their blood sugars rise if sugar alcohols are eaten in uncontrolled amounts. As the use of sugar alcohols in no- and low- carbohydrate foods has grown, increased consumer experience and awareness of these undesirable side effects has dampened enthusiasm to adopt sugar alcohol alternatives, stifling market expansion.

The problems with sugar alcohols are amplified by the widespread incidence of digestive disorders in the United States. The intestinal ecosystem plays an important role in normal gut function and maintaining host health. The host is protected from attack by potentially harmful microbial microorganisms by the physical and chemical barriers created by the gastrointestinal epithelium. The cells lining the gastrointestinal epithelium and the resident microbiota are two partners that properly and/or synergistically function to promote an efficient host system of immune defense. The gastrointestinal cells that make up the epithelium provide a physical barrier that protects the host against the unwanted intrusion of microorganisms into the gastrointestinal ecosystem, and against the penetration of harmful microorganisms which usurp the cellular molecules and signaling pathways of the host to become pathogenic. One of the basic physiological functions of the resident microbiota is that they function as a barrier against microbial pathogens.

The undesirable symptoms that result from consuming sugar alcohols are evidence that sugar alcohols effectively disrupt the intestinal ecosystem. Other than the obvious discomfort and inconvenience, intake of sugar alcohols can produce devastating diarrhea, resulting in serious electrolyte disturbances and dehydration. Such a disruption not only increases the potential of compromising competent immune surveillance, recognition and response activities, but can have a dangerous systemic impact. An unhealthy gastrointestinal (GI) tract environment is more vulnerable to inflammatory bowel disease and infestation by pathogenic organisms, including E. coli, clostridium difficile, rotavirus and H. pylori that further impair GI mucosal health. Primary among the many factors causing under nutrition, especially in old age, is altered function of the GI mucosa, which leads to heightened digestive sensitivities and specific malabsorption problems. These processes can often be seen in the elderly, as the normal composition and metabolic activities of the microbiota change with age in some individuals. Among their many functions, bifidobacteria and bacteroides are important for the production of short chain fatty acids and the breakdown of saccharides in the colon. Additionally, the widespread use of antibiotics indiscriminately alters healthy gut flora, impairing the production of the B vitamins such as, for example, folate and biotin, and short chain fatty acids important for overall health.

In 2002, the Center for Disease Control reported that people in the United States made 35.1 million visits to doctor's offices for digestive system symptoms and that 15.6 million adults were diagnosed with ulcers. Statistics from the National Digestive Disease Clearing House of the NIH indicate that digestive diseases affect 60-70 million Americans. Further, as much as 20-25% of the U.S. population has difficulty digesting milk because of its lactose content (characterized both as lactose intolerance and lactose maldigestion.) Lactose is also contained in chocolates made with milk. Sugar alcohols have been widely incorporated into milk chocolates to meet the demands of people wanting sugar-free candy, contributing lactose to GI tract insult and compounding the extent of digestive problems caused by sugar alcohols alone.

Attempts to inhibit the laxative effect of sugar alcohols by modifying them and/or using various agents to accelerate their breakdown have proven unsuccessful. Facilitating rapid breakdown of sugar alcohols would negate the low “net” carbohydrate impact benefit and reinstate a higher glycemic impact, regenerating Syndrome X and related insulin resistant complications. Further, attempts to alter hygroscopicity in an effort to reduce the laxative effect of sugar alcohols have also proven difficult and frustrating.

Various anecdotal reports and clinical observations note the severity of the laxative effect of sugar alcohols as being proportional to the quality of health and function of the GI tract. That is, the weaker the GI tract, the greater the digestive dysfunction and more severe the laxative effect of sugar alcohols. Lactitol, for example, has been shown to force an undesirably lower (more acidic) pH in parts of the colon. Increased volume and transit force of fluids in the bowel and alterations in colonic pH by sugar alcohols can reduce microbial adhesion to gut lumen and mucus and significantly alter the composition and effects of gut bacteria. Also, early animal studies indicate that maltitol is degraded by gut bacteria at sites distant from the absorptive area. R. Lian-Loh, G. G. Birch, M. E. Coates, The Metabolism of Maltitol in the Rat, Br. J. Nutr. 48:477-81 (1982). Sugar alcohols clearly affect changes in the physiology of the intestinal epithelium, which can alter the adhesive ability of Lactobacillus acidophilus. Lactobacillus acidophilus is a probiotic, or “healthy” bacteria that is present in the intestinal tract that protects against the entrance and proliferation of unhealthy organisms that can cause disease. As such, sugar alcohols can burden even healthy GI tract homeostasis and impair digestive function, resulting in the reported undesirable side effects. Therefore, introducing sugar alcohols into an already suboptimal ecosystem appears to catalyze further immune distress, inflammatory events, and the potential for devastating diarrhea in a dose dependent manner.

In view of the widespread use and benefits of sugar alcohols, a product that could reduce the adverse gastro-intestinal effects could contribute to the safety, efficacy and expanded use of sugar alcohols. Therefore, a need exists for a composition that prevents or treats symptoms of gastro-intestinal distress, especially those caused by the consumption of sugar alcohols. The present invention is a novel formula that uniquely, safely and naturally promotes increased resistance to the laxative effects of sugar alcohols used as sugar substitutes in no- and low-carbohydrate candies, snacks, foods and beverages. This novel technology combines safe and synergistic ingredients that promote healthy efficient digestive function, diminishing the laxative and flatulent effects. The technology of the present invention employs a novel approach to improving GI tract health and function, significantly increasing tolerance to and handling of gastrointestinal effects, especially those caused by consumption of sugar alcohols.

### SUMMARY OF THE INVENTION

The invention relates to compositions and methods for prevention or treatment of symptoms of gastrointestinal distress, including, but not limited to, diarrhea, loose or watery stools, inflammation, stomach and abdominal gas and/or pain, stomach rumblings, excessive belching, colic, flatulence, bloating, nausea and motion sickness. More particularly, the invention relates to compositions for prevention or treatment of symptoms of gastrointestinal distress and other symptoms caused by sugar alcohols, including, but not limited to, diarrhea, loose or watery stools, inflammation, stomach and abdominal gas and/or pain, stomach rumblings, excessive belching, colic, flatulence, bloating, nausea, motion sickness, heart burn and headaches.

A composition in accordance with the invention includes at least one enzyme, at least one probiotic organism, and simethicone. The at least one enzyme includes, without limitation, a proteolytic enzyme, beta-glucanase, xylanase, pectinase, phytase, alpha-galactosidase and combinations thereof. The at least one probiotic organism includes, without limitation, Lactobacillus acidophilus, Lactobacillus sporogenes, Lactobacillus rhamnosus, Bifidobacterium longum and combinations thereof. The probiotic organisms may be microencapsulated. The composition may additionally include an oxygen-coordinated chromium polynicotinate.

The invention further comprehends a method for preventing gastro-intestinal distress including consuming the above composition prior to consuming a food product including at least one sugar alcohol such as, for example, mannitol, sorbitol, xylitol, lactitol, isomalt, maltitol, hydrogenated starch hydrolysates, and combinations thereof. The composition may be consumed immediately prior to or simultaneously with, but not more than 20 minutes prior to consumption of the food product. The composition may decrease severity of at least one gastro-intestinal stress condition such as, for example, loose or watery stool, constipation, gas, bloating, burping, abdominal pain, stomach pain, heartburn, headache and combinations thereof, by at least about 50%.

### DETAILED DESCRIPTION OF THE INVENTION

The present invention relates to compositions and methods for the prevention and treatment of gastro-intestinal distress. More particularly, the present invention relates to compositions and method for the prevention and treatment of gastrointestinal distress caused by the consumption of sugar alcohols. Such gastro-intestinal distress symptoms include diarrhea, loose or watery stools, inflammation, stomach and abdominal gas and/or pain, stomach and bowel rumblings, increased belching, colic, flatulence, bloating, nausea, heart burn, motion sickness and headaches. These symptoms are often referred to as sugar alcohol-induced gastro-intestinal distress (SAIGID).

A composition for the treatment and/or prevention of gastro-intestinal distress includes at least one enzyme, at least one probiotic organism, and simethicone. Advantageously, the composition may be consumed immediately prior to or about 1 minute prior to and not more than about 20 minutes prior to the consumption of a food product including at least one sugar alcohol. In one embodiment, the composition may be consumed immediately prior to or simultaneously with consumption of a sugar alcohol-containing food product. Advantageously, a dosing regimen may include consuming the composition immediately prior to consumption of a sugar alcohol-containing food product which may reduce the severity of at least one gastro-intestinal stress condition such as, for example, loose or watery stools, constipation, gas, bloating, burping, abdominal pain, stomach pain, heartburn, headache or a combination thereof, by at least about 50%.

The food product may include at least one sugar alcohol such as, for example, mannitol, sorbitol, xylitol, lactitol, isomalt, maltitol, hydrogenated starch hydrolysates and combinations thereof. The food product may be, for example, a candy, a chocolate, a snacks food, a beverage or a bakery product.

Suitably, the at least one enzyme may include a proteolytic enzyme, beta-glucanase, xylanase, pectinase, phytase, alpha-galactosidase and combinations thereof. The enzymes in the composition provide a variety of functions to protect and treat against gastro-intestinal distress symptoms.

Proteolytic enzymes, also known as proteases, function to digest proteins in food. They are responsible for breaking down proteins and for the utilization of proteins. Proteases specifically refer to a group of enzymes whose catalytic function is to hydrolyze peptide bonds of proteins. Proteases differ in their ability to hydrolyze various peptide bonds and each type of peptide bond has a specific kind of peptide bond it breaks.

Beta-glucanase breaks down polysaccharides and fibers known as beta glucans. Xylanase hydrolyzes xylans, which are indigestible components of plant fibers and breaks down the sugar xylose.

Pectinase breaks down carbohydrates called hemi-celluloses, which are found in plant foods.

Phytase breaks down the undigestible phytic acid (phytate) portion in grains and oil seeds.

Alpha-galactosidase acts by cutting the glycosidic bond between the sugar galactose and another sugar molecule, not including galactose. It is an enzyme derived from Aspergillus niger and breaks down oligosaccharide linkages, which humans cannot digest and also allows patients to absorb single component sugar residues. Animal and human studies demonstrate that alpha-galactosidase improves intestinal bacterial populations and improves carbohydrate digestibility. B. Pan, D. Li, X. Piao, L. Zhang, L. Guo, Effect of Dietary Supplementation with Alpha-Galactosidase Preparation and Stachyose on Growth Performance, Nutrient Digestibility and Intestinal Bacterial Populations of Piglets, Arch Tierernahr 56:327-37 (2002). In addition, oral alpha-galactosidase solution has been shown to be moderately efficacious for the prophylaxis of gastrointestinal intolerance of oligosaccharides and has been marketed exclusively to prevent flatus and other gastro-intestinal symptoms resulting from a high-fiber diet. T. G. Ganiats, W. A. Norcross, A. L. Halverson, P. A. Burford, L. A. Palinkas, Does Beano Prevent Gas? A Double-blind Crossover Study of Oral Alpha-Galactosidase to Treat Dietary oligosaccharide Intolerance, J. Fam. Pract. 39:441-45 (1994). These enzymes are used in combination to prevent and treat gastro-intestinal symptoms through their specific functions. Alpha-galactosidase is uniquely applied specifically to improve gastro-intestinal tract competence and responsiveness to the ingestion of sugar alcohols.

Suitably, the at least on probiotic organism may include Lactobacillus acidophilus, Lactobacillus sporogenes, Lactobacillus rhamnosus, Bifidobacterium longum or combinations thereof.

Probiotic organisms, also known as “healthy” bacteria, are useful for a variety of functions, are present in the gastrointestinal (GI) tract, and protect against the entrance and proliferation of unhealthy organisms that can cause disease. An aberrant GI tract environment can induce undesirable oxidative stress and inflammatory sequela, irritating normal GI tract function. Probiotics exert a number of positive influences that contribute to improved gut health and immune system function. Lactobacillus acidophilus and Bifidobacterium longum have been shown to possess antioxidant activity. Although Bifidobacteria longum represent only 3-6% of the adult fecal flora, their presence has been associated with beneficial health effects, such as prevention of diarrhea, amelioration of lactose intolerance, and immunomodulation. Earlier studies examined Bifidobacteria longum to determine the relationship between their sensitivity to oxygen and oxygen metabolism. Activities of reduced NAD-oxidase and reduced NAD-peroxidase were inversely correlated with their sensitivities to oxygen. These observations are compatible with the hypothesis that reduced NAD-oxidase and reduced NAD-peroxidase in Bifidobacterium longum species play a role in prevention of oxygen toxicity, important for anaerobe viability in the GI tract. S. Shimamura, F. Abe, N. Ishibashi, H. Miyakawa, T. Yaeshima, Bifidobacterium Species, J. Dairy Sci 75:3296-3306 (1992).

Lactobacillus sporogenes produces beta galactosidase, also known as lactase, effective for digesting lactose in milk products.

Bifidobacterium longum contains numerous glycosyl hydrolases, enzymes that break down various saccharides. These sometimes novel glycosyl hydrolases appear to attack a wide spectrum of more complex, less common linkages found in plant polymers such as hemicelluloses, arabinogalactans, arabinoxylans, gums, inulins, galactomannans, and branched starches. In addition, animal research suggest that dietary supplementation with Bifidobacterium longum could provide benefits against enteric infection. This protective effect against a pathogenic challenge may be due to a reduced inflammatory response, mediated by the probiotic treatment. A. M. Silva, F. H. Barbosa, R. Duarte, L. Q. Vieira, R. M. Arantes, J. R. Nicoli, Effect of Bifidobacterium longum Ingestion on Experimental Salmonellosis in Mice, J. Appl. Microbiol 97:29-37 (2004). In addition, a major cause of diarrhea in infants and adults is due to the presence and binding of toxigenic E. coli to common bacterium-binding structures in the GI tract. Bifidobacterium longum has been shown to release a proteinaceous factor in culture that prevents binding of E. coli to such structures in the intestinal mucosa, demonstrating an important anti-infective benefit. Proinflammatory cytokines, such as tumor necrosis factor (TNF)-alpha and interleukin (IL)-1beta, up-regulate globotriaosylceramide (Gb3) expression, increase sensitivity to Vero cytotoxins (VCT; produced by E. coli), and enhance VCT action in developing hemorrhagic colitis and other GI tract diseases. Research corroborates that Bifidobacterium longum inhibits binding of E. coli food-born toxins to bacterial binding sites (i.e. globotriaosylceramide (Gb3)) in the gut epithelium. In addition, catalysts of inflammatory sequela (i.e. cytokine, TNF-alpha, and IL-1beta levels) in sera and expression of their mRNA were decreased, and expression of Gb3 in renal tubular epithelial cells was reduced in mice treated with Bifidobacterium longum. S. H. Kim, S. J. Yang, H. C. Koo, W. K. Bae, J. Y. Kim, J. H. Park, Y. J. Back, Y. H. Park, Inhibitory Activity of Bifidobacterium longum HY 8001 Against Vero Cytotoxin of Escherichia coli O157:H7, J. Food Prot 64:1667-1673 (2001).

Intestinal microbial populations are deranged in hemodialysis patients, evidenced by an increase in aerobic bacteria such as Escherichia coli and a decrease in anaerobic bacteria such as Bifidobacterium longum. Serum levels of indoxyl sulfate are increased markedly in hemodialysis patients and cannot be reduced efficiently by hemodialysis because of its albumin binding. Intake of Bifidobacteria has been shown to restore disturbed microflora to normal. Bifidobacteria are important to ferment carbohydrates, producing acetic acid and lactic acid, which effectively inhibits putrefaction in the intestines. However, due to exposure to gastric juices, Bifidobacteria in most commercial products cannot usually survive to reach the intestines. Microencapsulation of organisms (like Bifidobacterium longum) prevents inactivation by acidic gastric juice and facilitates passage to the intestines where they are effectively activated.

Oral administration of Bifidobacterium longum in a gastro-resistant seamless capsule to hemodialysis patients has been shown to effectively decrease the pre-hemodialysis serum levels of homocysteine, indoxyl sulfate, and triglyceride. The reduction in the serum level of homocysteine is mainly attributable to the supply of folate produced by Bifidobacterium longum in the human intestines. F. Takayama, K. Taki, T. Niwa, Bifidobacterium in Gastro-resistant Seamless Capsule Reduces Serum levels of Indoxyl Sulfate in Patients on Hemodialysis, Am J Kidney Dis 41 (3 Suppl 1):S142-S145 (2003); K. Taki, F. Takayama, T. Niwa, Beneficial Effects of Bifidobacteria in a Gastro-Resistant Seamless Capsule on Hyperhomocysteinemia in Hemodialysis Patients, J Ren Nutr 15:77-80 (2005).

Liver cirrhosis is characterized by chronic bile retention, acute inflammation, excess fat accumulation, extensive liver tissue damage and GI tract dysfunction. Patients with liver cirrhosis have varying degrees of imbalance of the intestinal flora as shown by a decrease in the Bifidobacterium count. Interestingly, the severity of the imbalance is proportional to and corresponds with the level of liver dysfunction. Probiotics effectively increased the Bifidobacterium count and reduced the level of fecal pH and fecal and blood ammonia in certain populations.

An unhealthy GI tract provides an opportunistic environment for infestation and proliferation of pathogenic bacteria. Chronic infection with Helicobacter pylori (H. pylori) is responsible for significant impairment of healthy gastric function and has been shown to cause peptic ulcers, gastric cancer and lymphoma. Lactobacillus acidophilus DDS-1J was shown to exert a growth inhibitory effect on H. pylori at a ratio of 1:1 or higher in vitro. A. Chatterjee, T. Yasmin, D. Bagchi, S. J. Stohs, The Bacterial Effects of Lactobacillus acidophilus, Garcinol and Protykin Compared to Clarithromycin, on Helicobacter pylori, Mol Cell Biochem 243:29-35 (2003). In addition, Lactobacillus acidophilus was found to have an inhibitory effect on H. pylori isolated from peptic ulcer patients. Approximately an equal density of Lactobacillus acidophilus on H. pylori had the most favorable effect.

Mucosal immuno-stimulation by lactic acid bacteria varies depending upon the strain being studied and the sites of mucosal contact and internalization in the gut. Some of the lactic acid bacteria increase the inflammatory immune response and others enhance the level of secretory antibody (S-IgA). The induction of the gut mucosal immune response is dependent on the antigen interacting with the M cells of Peyer's patches and with the immune cells associated with this lymphoid organ. The pathways by which lactic acid bacteria are internalized were assessed in an animal model. Lactobacillus acidophilus was found to interact with Peyer's patches, epithelial cells of the small intestine and epithelial cells of the large intestine, providing evidence of multiple pathways of internalization and immunostimulation. Certain lactic acid bacteria are able to induce specific secretory immunity and others will enhance the inflammatory immune response. Modulation of intestinal epithelial cell (IEC) cytokine production has the potential to profoundly affect the mucosal microenvironment, influencing the immune response to pathogens and other ingested antigens. As a participant in the mucosal immune response, the IEC must respond to a variety of stimuli, including lactic acid bacteria consumed in the diet. Strains of Lactobacillus rhamnosus and Lactobacillus acidophilus suppressed the production of the chemokine RANTES by stimulated HT-29 IEC, although the magnitude of this suppression varied depending on the nature of the bacterial growth medium. Similarly, specific strains showed growth condition-dependent suppression of HT-29 interleukin-8 (IL-8) production. Strain-dependent effects were also seen for the suppression of tumor necrosis factor alpha (TNF-alpha) and transforming growth factor beta (TGF-beta) production. Different strains were found to have differing abilities to interact with IEC, with Lactobacillus rhamnosus R0011 being the strain that generally had the most extensive effects on HT-29 cytokine production and also bound to HT-29 IEC most effectively. T. D. Wallace, S. Bradley, N. D. Buckley, J. M. Green-Johnson, Interactions of Lactic Acid Bacteria with Human Intestinal Epithelial Cells: Effects on Cytokine Production, J Food Prot 66:466-72 (2003).

Oral administration of milk fermented with Lactobacillus acidophilus (L-92) resulted in a statistically significant improvement of perennial allergic rhinitis. Ocular symptom-medication scores of patients in the L-92 intervention group tended to improve compared with those in the placebo group. In addition, clear decreases of the scores of swelling and color of the nasal mucosa were observed in the L-92 intervention group at 6 and 8 weeks after the start of ingestion of fermented milk, providing compelling evidence of improved host immune function and tolerance to common antigens. Y. Ishida, F. Nakamura, H. Kanzato, D. Sawada, H. Hirata, A. Nishimura, O. Kajimoto, S. Fujiwara, Clinical Effects of Lactobacillus acidophilus Strain L-92 on Perennial Allergic Rhinitis: A Double-Blind, Placebo-Controlled Study, J Diary Sci 88:527-33 (2005).

The relative proportion of Polymorphonucleocyte cells (PMNs) showing phagocytic activity increased following consumption of Lactobacillus rhamnosus HN001 in either Low Fat Milk or Lactose Hydrolyzed Low Fat Milk and resulted in a dramatic increase in the level of NK cell tumor killing activity by 71% and 147% respectively. In most cases these levels declined following cessation, but remained above baseline. Dietary consumption of Lactobacillus rhamnosus HN001, in a base of low-fat milk or lactose-hydrolyzed low-fat milk, appears to enhance systemic cellular immune responses and may be useful as a dietary supplement to boost natural immunity. Ying-H. Sheih, Bor-L. Chiang, Ling-H. Wang, Chuh-K. Liao, Harsharnjit S. Gill, Systemic Immunity-Enhancing Effects in Healthy Subjects Following Dietary Consumption of the Lactic Acid Bacterium Lactobacillus rhamnosus HN001, J Am Coll Nutr 20:149-156 (2001).

Lactobacillus rhamnosus produces an extracellular polysaccharide composed of D-glucose and D-galactose in a molar ratio of 2:3. While glucose is targeted primarily at energy production and to a lesser extent at nourishing glucose sensing neurons in the brain, galactose is an important monosaccharide for a range of biological roles. Almost all immune complexes are glycoproteins. Galactose has been shown to be critical in the formation of IgG and Type II Collagen. Unavailable galactose has been shown to disrupt formation of the correct 3 dimensional structure of glycoproteins requiring it (e.g. IgG and Type II Collagen), impairing the structure and function of those molecules. Both impaired IgG function and malformation of Type II Collagen have been implicated in the etiology of rheumatoid arthritis and the inflammatory sequela of Osteoarthritis (OA). D. Bagchi et al., Effects of Orally Administered Undernatured Type II Chicken Collagen Against Arthritic Inflammatory Diseases: A Mechanistic Exploration, Int J Clin Pharm Res 23:101-110 (2002). As such, Lactobacillus rhamnosus can help reduce inflammatory sequela and contribute to immune competence by different pathways than previously noted. In fact, in a 12 month pilot study on the effects of Lactobacillus rhamnosus for rheumatoid arthritis, more subjects reported improvement in well being after taking Lactobacillus rhamnosus. K. Hatakka, J. Martio, M. Korpela, M. Herranen, T. Poussa, T. Laasanen, M. Saxelin, H. Vapaatalo, E. Moilanen, R. Korpela, Effects of Probiotic Therapy on the Activity and Activation of Mild Rheumatoid Arthritis—A Pilot Study, Scand J Rheumatol 32: 211-215 (2003). Further, Lactobacillus rhamnosus has been shown to possess a clear anti-inflammatory effect in gastrointestinal disease and allergy. Lactobacillus rhamnosus has also demonstrated an ability to prolong intestinal epithelial cell viability, inhibiting cytokine induced apoptosis and related inflammatory events.

Live Streptococcus thermophilus and Lactobacillus acidophilus interact with intestinal epithelial cells to protect them from the deleterious effect of entero-invasive E. coli via mechanisms that include interference with pathogen adhesion and invasion. Probiotics likely also enhance the barrier function of naive epithelial cells not exposed to any pathogen, promoting healthy gut function. In children 6-24 months, live lactobacillus supplementation suppressed pneumonia and decreased bronchitis in undernourished as well as in normal children during a 3 month period spanning autumn to winter. M. E. Rio, Beatriz L. Zago, H. Garcia, L. Winter, The Nutritional Status Change the Effectiveness of a Dietary Supplement of Lactic Acid Bacteria on the Emerging of Respiratory Tract Diseases in Children, Arch Latinoam Nutr 52: 29-34 (2002). These effects demonstrate the role of Lactobacillus acidophilus in promoting improvements in overall health by boosting immune competence thus reducing the involvement of immune mediated inflammatory events. Other research demonstrated that strains of Lactobacillus rhamnosus and Lactobacillus acidophilus provided a safe means for reducing H. pylori colonization and bacterial-induced inflammation in mice. K. C. Johnson-Henry, D. J. Mitchell, Y. Avitzur, E. Galindo-Mata, N. L. Jones, P. M. Sherman, Probiotics Reduce Bacterial Colonization and Gastric Inflammation in H. pylori-infected Mice, Dig Dis Sci 49:1095-1102 (2004).

Simethicone provides antifoaming benefits to aid in “bubble reduction” during GI distress. The drug acts on the surface of bubbles by reducing the surface tension and thereby disrupting or breaking the bubble.

In one embodiment, the composition may include about 20 to about 40 composition weight percent of at least one microencapsulated probiotic organism and about 60 to about 80 composition weight percent of a blend of simethicone and at least one enzyme.

In a further embodiment, the composition may include about 20 to about 40 composition weight percent of a blend of microencapsulated probiotic organisms including L. sporogenes, L. acidophilus, L. rhamnosus and B. longum. The composition may also include about 60 to about 80 composition weight percent of a blend of simethicone, alpha-galactosidase, protease (papain), glucanase, xylanase, pectinase and phytase.

Advantageously, the composition may further include a chromium-containing compound, such as an oxygen-coordinated chromium polynicotinate. Suitably, the chromium-containing compound may be included in the composition in a concentration sufficient to provide about 50 to about 200 micrograms or about 100 micrograms of elemental chromium per dose. One oxygen-coordinated chromium polynicotinate compound suitable for use in the composition may be obtained under the registered trademark ChromeMate® from InterHealth Nutraceuticals, Inc. of Benicia, Calif.

Chromium deficiency is thought to contribute to glucose intolerance and unhealthy blood lipid profiles. The primary function of chromium is to potentiate the effects of insulin and thereby enhance glucose, amino acid and fat metabolism. Chromium supplements have been purported to increase muscle mass and decrease body fat. A lack of chromium can impair insulin function, also inhibiting protein synthesis and energy production. Chromium deficiency can lead to type II diabetes and even heart disease

The alterations in the insulin metabolism due to increased insulin resistance can lead to many forms of hypertension and increased blood pressure. Hypertension, a major public health problem, becomes more prevalent during aging. It is commonly accepted that blood pressure (BP) increases steadily in most individuals with aging. Preuss et al. (1997) has shown that hypertension is found in 50% or more individuals above age 55 and 63% of those aged 65-74. The rate is as high as 76% among African Americans over 65. Chromium has been shown to reduce levels of harmful LDL cholesterol, a form of cholesterol that increases with age and leads to heart disease and hypertension, and to increase beneficial HDL cholesterol. E. G. Offenbacher, F. X. Pi-Sunyer, Beneficial Effect on Chromium-rich Yeast on Glucose Tolerance and Blood Lipids in Elderly Subjects, Geriatric Nephrology and Urology 6:169-179 (1997).

Exogenous insulin requirements decrease from 200 micrograms daily to 0 micrograms following chromium supplementation with a normalization of blood glucose. In the last five years, chromium has been shown to play a role in Type II diabetes mellitus, gestational diabetes, steroid-induced diabetes and glucose tolerance. In summary, supplementation with chromium had beneficial effects on persons with varying degrees of glucose intolerance, ranging from marginal glucose intolerance to uncontrolled diabetes. Chromium also reduced the amount of insulin required for persons with diabetes. There have been no reported side effects with chromium supplementation. R. A. Anderson, Chromium and Diabetes, Nutrition 15: 720-22 (1999); R. A. Anderson, Chromium, Glucose Intolerance and Diabetes, Journal of the American College of Nutrition 17:548-555; R. A. Anderson et al., Elevated Intakes of Supplemental Chromium Improves Glucose and Insulin Variables in Individuals with Type 2 Diabetes, Diabetes 46:1786-1791 (1997); H. G. Preuss, R. A. Anderson, Chromium Update: Examining Recent Literature, Current Opinion on Clinical Nutrition and Metabolic Care, 6:509-512 (1998); A. Ravina et al., Control of Steroid-Induced Diabetes with Supplemental Chromium, The Journal of Trace Elements in Experimental Medicine 12:375-78 (1999).

A study at Auburn University showed that a composition containing an oxygen-coordinated chromium polynicotinate reduced LDL cholesterol in humans by an average of 14%. R. G. Lefavi et al., Lipid Lowering Effect of a Dietary Chromium (III)-Nicotinic Acid Complex in Male Athletes, Nutrition Research 13:239-49 (1993).

Preuss et al. (1997) confirmed that chromium supplementation can overcome sucrose induced blood pressure elevation in spontaneously hypertensive rats. Preuss et al. (1995) have suggested that essential hypertension may be due to insulin perturbations and as high dose chromium supplementation seems non-toxic, chromium may prove to be a useful means to lower blood pressure (BP) in some essential hypertensives as well as diabetic hypertensives. Preuss and Anderson recently noted that chromium supplementation may prove to be the most useful means to prevent or treat type II diabetes mellitus and various cardiovascular disorders. Researchers at the University of Texas, Austin showed that young obese women taking oxygen-coordinated chromium nicotinate while exercising resulted in significant weight loss and lowered insulin response to an oral glucose load, while those taking chromium picolinate resulted in significant weight gain. Niacin bound chromium given to modestly dieting, exercising African-American women caused a significant loss of fat and sparing of muscle compared to placebo. Chromium supplementation amplifies insulin receptor tyrosine kinase activity, which explains, in part, the relationship between chromium and its effects in diabetes. Chromium further reduces vascular smooth muscle calcium loads and thus reduces peripheral vascular resistance in insulin-resistant states. Hence, chromium supplementation may prove to be a useful means to prevent or treat Type II diabetes mellitus and various cardiovascular disorders. Recently, the U.S. Department of Agriculture (USDA) found that many middle-age diabetics could overcome their symptoms by taking a chromium supplement. The USDA's findings suggest that very low chromium intakes may be putting millions of Americans on the road to diabetes (and high blood cholesterol) and that the process could be reversed by supplementing with chromium. V. Crawford et al., Effects of Niacin-Bound Chromium Supplementation on Body Composition in Overweight African-American Women, Diabetes, Obesity and Metabolism 1:1-7 (1999); H. G. Preuss and R. A. Anderson, Chromium Update: Examining Recent Literature, Current Opinion on Clinical Nutrition and Metabolic Care 6:509-512 (1998); R. G. Lefavi et al., Lipid Lowering Effect of a Dietary Chromium (III)-Nicotinic Acid Complex in Male Athletes, Nutrition Research 13:239-249 (1993); H. G. Preuss et al., Effects of Different Compounds on Blood Pressure and Lipid Peroxidation in Spontaneously Hypertensive Rats, Nephrology 47:325-330 (1997); H. G. Preuss et al., Effects of Chromium and Guar on Sugar Induced Hypertension in Rats, Clinical Nephrology 44:170-177 (1995).

Chromium has been shown to decrease the portion of systolic blood pressure elevated by high sucrose intake as shown previously, but continuously high levels of sucrose ingestion, without a concomitant increase in chromium, have been shown to eventually overcome this.

The composition herein described may be provided in any suitable from including, but limited to, capsules, caplets, tablets and powders. Additionally, the composition may further include one or more inert and/or inactive ingredients such as, for example, bulking agents like maltodextran and other processing aids.

### EXAMPLES

### Example 1

### Effects Composition on Gastro-Intestinal Distress when Consuming Sugar Alcohols

A total of twenty-two (22) study participants were randomly selected who agreed to participate in a brief study on the extent to which taking the composition described herein could reduce gastro-intestinal distress from consuming foods containing sugar substitutes, particularly, sugar alcohols. All 22 participants completed the screening questionnaire shown in Table 1 indicating the extent to which the nine (9) listed gastro-intestinal stress conditions were typically a problem. Respondents indicated which conditions were a problem and the frequency (seldom, occasionally, frequently and every time) with which the problem occurred. Four people indicated that they traditionally had no reaction to the listed conditions and were excluded from the study. From a post-study interview, it was determined that the four people excluded from the study did have one or more of the listed conditions, but were reluctant to participate in the study and were therefore not included in the study. The remaining 18 reported significant gastro-intestinal stress problems and agreed to take food containing sugar alcohols with and without the composition described herein. Using a scale of 1-4 for each of the 9 items, a total score was calculated for each subject. Each Participant's self-ranked typical gastro-intestinal stress level (Typical GIS) is listed in Table 2, below.

TABLE 1
Gastro-Intestinal Stress Questionnaire

| | |
|---|---|
| 1 | Loose or watery stools |
| 2 | Constipation |
| 3 | Gas |
| 4 | Bloating |
| 5 | Burping |
| 6 | Abdominal Pain |
| 7 | Stomach Pain |
| 8 | Heartburn |
| 9 | Headaches |

1 = Seldom
2 = Occasionally
3 = Frequently
4 = Every time

Participants then consumed an ASHER'S Sugar-free Liquid Caramel candy bar that weighed 1.65 ounces and contained 27 grams of maltitol. On the day following the consumption of the bar, each participant rated the level of gastro-intestinal stress they experienced when eating the candy bar using the 9 items listed on the questionnaire. Each Participant's self-ranked gastrointestinal stress level (Candy GIS) is listed in Table 2, below.

Participants were provided with capsules containing a total of 600 mg of a composition in accordance with the invention. In particular, the capsules contained 600 mg of Composition T1713 which included 426 mg of simethicone and digestive enzymes and 138 mg of microencapsulated probiotics. The digestive enzymes included alpha-galactosidase, protease (papain), glucanase, xylanase, pectinase and phytase. The microencapsulated probiotic organisms included a blend of L. sporogenes, L. acidophilus, L. rhamnosus, and B. iongum.

Participants consumed 600 mg of the composition described herein immediately before consuming another ASHER'S candy bar. On the following day, participants completed the gastro-intestinal stress questionnaire and rated the percentage of improvement in gastro-intestinal stress when consuming the composition with the candy bar. Each Participant's self-ranked gastro-intestinal stress level (Composition GIS) is listed in Table 2, below.

Reductions in gastro-intestinal stress level (Decrease in GIS) for all 18 participants after consuming the composition immediately before consuming the candy bar containing the sugar alcohols is listed in Table 2, below. All subjects reported a reduction in gastrointestinal stress when taking the composition with an average estimated reduction in symptoms (Self-Reported % Decrease) of 67%.

TABLE 2
Study Results

| | Screen- | | | Compo- | De- | Self- |
|---|---|---|---|---|---|---|
| Subject | ing | Typical | Candy | sition | crease | Reported |
| No. | Data | GIS | GIS | GIS | in GIS | % Decrease* |
| 1 | 4 | 8 | 12 | 2 | 10 | 75 |
| 2 | 2 | 5 | 15 | 1 | 14 | 60 |
| 3 | 3 | 8 | 15 | 2 | 13 | 80 |
| 4 | 3 | 6 | 10 | 0 | 10 | 75 |
| 5 | 4 | 6 | 11 | 0 | 11 | 80 |
| 6 | 3 | 6 | 4 | 2 | 2 | 40 |
| 7 | 2 | 7 | 16 | 1 | 15 | 100 |
| 8 | 3 | 6 | 6 | 1 | 5 | 80 |
| 9 | 3 | 4 | 8 | 2 | 6 | 75 |
| 10 | 6 | 8 | 14 | 4 | 10 | 65 |
| 11 | 5 | 5 | 8 | 2 | 6 | 70 |
| 12 | 4 | 7 | 11 | 0 | 11 | 95 |
| 13 | 2 | 6 | 8 | 2 | 6 | 60 |
| 14 | 4 | 16 | 17 | 1 | 16 | 75 |
| 15 | 3 | 4 | 8 | 2 | 6 | 80 |
| 16 | 3 | 5 | 16 | 1 | 15 | 70 |
| 17 | 8 | 23 | 15 | 5 | 10 | 5 |
| 18 | 6 | 17 | 9 | 2 | 7 | 20 |
| Average | 3.8 | 8.2 | 11.3 | 1.7 | 9.6 | 67 |

A Calculated % Decrease was determined based on the difference between the Candy GIS and the Composition GIS (Decrease in GIS) divided by the Candy GIS. The average Calculated % Decease is about 85%. It was found that the Calculated % Decrease was statistically significant and consistent with the study participants' Self-Reported % Decrease.

A Change score was obtained for each participant by subtracting the GIS rating of the candy with and without the composition. A Relative percent change (Relchange) score was calculated for each participant by dividing the amount of change by the GIS rating without the composition. A null hypothesis of no treatment effect was tested with regard to the Change and with regard to the Relchange using the Wilcozon Rank Sum Test. Exact methods were used to determine the p-value. Pearson correlations were used to assess the relation between the candy, the composition, Change, Relative Change (Relchange), Screen, Typical and the reported percentage change as shown in Table 3.

TABLE 3
Pearson Correlations and p-values

| | Typical | Candy | Composition | | | |
|---|---|---|---|---|---|---|
| | GIS | GIS | GIS | Change | Relchange | Pchange |
| Screen | 0.75 | 0.13 | 0.65 | 0.08 | −0.32 | −0.64 |
| | (<0.001) | (0.60) | (0.003) | (0.76) | (0.20) | (0.004) |
| Typical GIS | | 0.36 | 0.51 | −0.19 | −0.16 | −0.68 |
| | | (0.14) | (0.03) | (0.46) | (0.52) | (0.002) |
| Candy GIS | | | 0.08 | −0.95 | 0.50 | 0.10 |
| | | | (0.76) | (<0.001) | (0.03) | (0.68) |
| Composition | | | | 0.24 | −0.74 | −0.65 |
| GIS | | | | (0.33) | (<0.001) | (0.004) |
| Change | | | | | −0.72 | −0.31 |
| | | | | | (<0.001) | (0.21) |
| Relchange | | | | | | 0.60 |
| | | | | | | (0.008) |

### Example 2

### Consumption of Isomalt-containing Food

Fifteen (15) of the participants in the above study agreed to also take 900 mg of the composition described herein immediately prior to consuming two ounces of sugar-free caramel popcorn containing 37 grams of isomalt. On the following day, participants were asked to report their overall level of gastrointestinal stress after consuming the popcorn. A total of 11 participants reported no gastro-intestinal stress after consuming the popcorn and 4 reported only mild to very mild flatulence.

### Example 3

### Triple-blind Efficacy Study—Stage 1

A total of 247 participants were randomly selected who agreed to participate in a study on the extent to which taking the composition described herein could reduce gastro-intestinal distress from consuming foods containing sugar substitutes, particularly, sugar alcohols. Each participant completed the screening questionnaire shown in Table 4 indicating, on a scale of 0-10, the extent to which the nine (9) listed gastro-intestinal stress conditions were typically a problem and their level of overall daily intestinal distress. Participants consumed an ASHER'S Sugar-free Liquid Caramel candy bar that weighed 1.65 ounces and contained 27 grams of maltitol. Twenty-four hours after consumption of the bar, each participant rated the level of gastro-intestinal stress they experienced when eating the candy bar using the 10 items listed on the questionnaire in Table 4.

TABLE 4
Efficacy Questionnaire

| | |
|---|---|
| 1 | Loose or watery stools |
| 2 | Constipation |
| 3 | Gas |
| 4 | Bloating |
| 5 | Burping |
| 6 | Abdominal Pain |
| 7 | Stomach Pain |
| 8 | Heartburn |
| 9 | Headaches |
| 10 | Overall intestinal discomfort |

None 0 . . . 1 . . . 2 . . . 3 . . . 4 . . . 5 . . . 6 . . . 7 . . . 8 . . . 9 . . . 10 High

Participants were randomly assigned to one of three groups as shown in Table 5. Particpants in Group A consumed 600 mg of Composition T1713, disclosed above, immediately prior to consuming an ASHER'S Sugar-free Liquid Caramel candy bar. Participants in Group B consumed 600 mg of a placebo containing an inert blend of cellouse immediately prior to an ASHER'S Sugar-free Liquid Caramel candy bar. Participants in Group C consumed 600 mg of Composition T1713C immediately prior to consuming an ASHER'S Sugar-free Liquid Caramel candy bar. Composition T1713C included 426 mg of a blend of simethicone, alpha-galactosidase, protease (papain), glucanase, xylanase, pectinase and phytase, 138 mg of a blend of microencapsulated probiotic organisms including L. sporogenes, L. acidophilus, L. rhamnosus and B. longum, and 100 micrograms of elemental chromium in the form of an oxygen-coordinated chromium nicotinate.

TABLE 5
Stage 1 Group Assignments

| Group | # of Participants |
|---|---|
| A | 81 |
| B | 71 |
| C | 105 |
| Total | 257 |

Twenty-four hours after consumption of the bar, each participant gastro-intestinal stress they experienced when eating the candy bar 10 items listed on the questionnaire in Table 4. Forty-eight hours after candy bar, each participant consumed 600 mg of the same composition they previously consumed immediately prior to consuming a second ASHER'S Sugar-free Liquid Caramel candy bar. Twenty-four hours after consumption of the second candy bar, each participant completed another 10 item efficacy questionnaire.

For each participant, an average of the 10 self-reported discomfort measures or rating from each questionnaire was calculated. Change scores were obtained by subtracting the rating after taking a candy bar with a tablet from the rating obtained after taking a candy bar without the tablet. Analysis of the change scores calculated from the questionnaire data revealed that there were no significant differences between the results obtained from participants consuming one of the two active products (Group A and Group C). Both Group A and Group C participants reported about a 60% improvement in gastro-intestinal distress compared to about a 10% improvement reported by Group B participants. Each active product was found to produce a six-fold improvement in discomfort over the placebo:
Group A vs. Group B: P=0.040; and
Group C vs. Group B: P=0.022.

### Example 4

### Triple-blind Efficacy Study—Stage 2

A total of 76 participants were randomly selected and randomly assigned to one of three groups as shown in Table 6, below. Participants in Group A consumed 600 mg of Composition T1713, disclosed above. Participants in Group B consumed 600 mg of a placebo containing an inert blend of cellulose. Participants in Group C consumed 600 mg of Composition T1713C, disclosed above. Each participant followed the same test protocol as in Stage 1.

TABLE 6
Stage 2 Group Assignments

| Group | # of Participants |
|---|---|
| A | 27 |
| B | 18 |
| C | 31 |
| Total | 76 |

Analysis of the Stage 2 data revealed almost identical changes in the three groups with no statistically significant differences between Group A and Groups C which consumed active products T1713 and T1713C, respectively.

Because the placebo and both active products produced almost exactly the same amount of change as in Stage 1, the data from the A and C groups in both stages were combined and compared to the combined data for the placebo groups, Group B. Combined Group A was found to have about a 60% improvement vs. an about 10% improvement for Combined Group B at P=0.014.

### Results

The studies revealed highly significant (P<0.001) changes in gastro-intestinal stress scores when consuming the products containing sugar alcohols after taking the composition described herein as compared to consuming these products without taking the composition as well as both safe and effective in reducing gastrointestinal discomfort.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

I claim:

1. A composition for the treatment or prevention of gastrointestinal distress, comprising:
at least one enzyme;
at least one probiotic organism; and
simethicone.

2. The composition of claim 1, wherein the at least one enzyme is selected from the group consisting of a proteolytic enzyme, beta-glucanase, xylanase, pectinase, phytase, alpha-galactosidase and combinations thereof.

3. The composition of claim 1, wherein the at least one probiotic organism is selected from the group consisting of Lactobacillus acidophilus, Lactobacillus sporogenes, Lactobacillus rhamnosus, Bifidobacterium longum and combinations thereof.

4. The composition of claim 1, wherein the at least one probiotic organism in microencapsulated.

5. The composition of claim 1, comprising about 20 to about 40 composition weight percent of at least one probiotic organism.

6. The composition of claim 1, comprising about 60 to about 80 composition weight percent of a blend of simethicone and at least one enzyme.

7. The composition of claim 1, further comprising an oxygen-coordinated chromium polynicotinate.

8. The composition of claim 7, wherein the oxygen-coordinated chromium polynicotinate is present in a concentration sufficient to provide about 50 to about 200 mcg of elemental chromium.

9. The composition of claim 1, comprising:
about 20 to about 40 composition weight percent of a blend of microencapsulated probiotic organisms including Lactobacillus acidophilus, Lactobacillus sporogenes, Lactobacillus rhamnosus and Bifidobacterium longum; and
about 60 to about 80 composition weight percent of a blend of enzymes including alpha-galactosidase, papain, glucanase, xylanase, pectinase and phytase.

10. The composition of claim 9, further comprising about 50 to about 200 mcg of elemental chromium.

11. The composition of claim 1, wherein the composition reduces severity of at least one gastro-intestinal stress condition selected from the group consisting of loose or watery stools, constipation, gas, bloating, burping, abdominal pain, stomach pain, heartburn, headache and combinations thereof.

12. The composition of claim 1, wherein the composition is taken prior to consumption of a food product comprising at least one sugar alcohol.

13. The composition of claim 12, wherein the at least one sugar alcohol is selected from the group consisting of mannitol, sobitol, xylitol, lactitol, isomalt, maltitol, hydrogenated starch hydrolysates and combinations thereof.

14. The composition of claim 12, wherein the food product is selected from the group consisting of candies, chocolates, snacks foods, beverages and bakery products.

15. A method for preventing gastro-intestinal distress, comprising consuming a composition including at least one enzyme, at least one probiotic organism and simethicone prior to consumption of a food product including at least one sugar alcohol.

16. The method of claim 15, wherein the composition is consumed immediately prior to consuming the food product.

17. The method of claim 15, wherein the composition is consumed between about 1 and about 20 minutes prior to consuming the food product

18. The method of claim 15, wherein the composition is consumed not more than about 20 minutes prior to consuming the food product

19. The method of claim 14, wherein a severity of at least one gastro-intestinal stress condition is reduced by at least about 50%.

20. The method of claim 19, wherein the at least one gastro-intestinal stress condition is selected from the group consisting of loose or watery stools, constipation, gas, bloating, burping, abdominal pain, stomach pain, heartburn, headache and combinations thereof.
